(19) United States
(12) Patent Application Publication
(10) Pub. No.: US 2005/0137573 A1
(43) Pub. Date: Jun. 23, 2005

(54) System, method, and communication hub for controlling external infusion device

(75) Inventor: Brian J. McLaughlin, Media, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Animas Corporation

(21) Appl. No.: 11/013,911

(22) Filed: Dec. 17, 2004

Related U.S. Application Data
(60) Provisional application No. 60/530,660, filed on Dec. 19, 2003.

(51) Int. Cl. A61M 31/00

(52) U.S. Cl. 604/504; 604/65

(57) ABSTRACT

A communication hub for controlling an external infusion device includes a first communication interface for transferring information from at least one external device to the communication hub, and a second communication interface for transferring the information received from the at least one external device from the communication hub to the external infusion device. The communication hub is configured to be connected to the external infusion device.

This application claims the benefit of priority under 35 U.S.C. § 119(e) of U.S. provisional application No. 60/530,660, filed on Dec. 19, 2003, the disclosure of which is incorporated herein by reference.

### BACKGROUND OF THE INVENTION

1. Field of the Invention

The present invention relates to a system, method, and communication hub for controlling an external infusion device. More particularly, the present invention relates to a system and method for regulating transport of fluid into a user, and a communication hub for carrying an external infusion device and for controlling an external infusion device based on information from one or more external devices.

2. Description of the Related Art

External infusion devices (e.g., pumps) may be used for delivering medication to users, such as insulin to diabetics. Portable external infusion devices may be attached to a user's belt, for example, or placed in a user's pocket. In external infusion devices delivering insulin, for example, the insulin may delivered via a cannula, inserted in subcutaneous tissue of the user.

Some conventional external infusion devices may communicate remotely with another information device, such as a controller that is physically separated from the external infusion device, for altering one or more functional settings of the external infusion device. In such conventional external infusion devices, communication circuitry necessary for providing this communication capability is located within the housing of the external infusion device itself. Incorporating the communication circuitry into the housing of the external infusion device, however, may create certain drawbacks.

One possible drawback relates to damage to the circuitry if, for example, the external infusion device is immersed in water. In order to render an external infusion device more resistant to water, it may be necessary to permanently seal the housing of the external infusion device. As a result, it may be impractical, if not impossible, to either adjust or repair the communication circuitry within the housing once the housing has been sealed.

Another possible drawback relates to governmental agencies, such as the Federal Communication Commission (FCC), requiring the use of specified radio frequency (RF) bands for wireless communication in medical devices. The FCC's required RF bands, however, are not necessarily the same as RF bands used in other countries. For example, an RF band frequency of 902 MHz, which is allowed for some medical device RF communication in the U.S., is not allowed in Europe. In Europe, an RF band frequency of 868 MHz is required for corresponding medical devices, instead of 902 MHz. Furthermore, the FCC and other regulatory agencies have indicated that they oppose allowing such medical devices to be capable of enabling the user to select appropriate RF band frequencies corresponding to the user's geographic location. The RF band frequency, therefore, is permanently preset in the U.S. by the manufacturer. Consequently, if a user of an external infusion device preset for RF transmission in the U.S. travels to Europe or elsewhere in the world, the external infusion device is likely to operate in violation of local regulations. Furthermore, the external infusion device may interfere with the operation of other devices operating in the same RF band.

Another possible drawback relates to a desire, for the user's comfort and convenience, to make external infusion devices as small as possible. For example, external infusion devices are often designed to be small enough to fit into a user's clothing pocket. In external infusion devices having RF circuitry located within such a small housing, the RF circuitry must also be small. The antenna may be so small, however, that the communication range of the circuitry is limited to an undesirable extent.

In light of the above-mentioned possible drawbacks, a desire exists for devices and methods for transferring information between external devices and an external infusion device that may overcome or alleviate one or more of the above-mentioned drawbacks.

### SUMMARY OF THE INVENTION

The present invention is directed to a system and a communication hub for transferring information between one or more external devices and an external infusion device, that may obviate one or more of the possible drawbacks associated with the limitations and disadvantages of the above-mentioned conventional devices.

In the following description certain aspects and embodiments will become evident. It should be understood that the invention, in its broadest sense, can be practiced without having one or more features of these aspects and embodiments, and that these aspects and embodiments are merely exemplary.

In one aspect, as embodied and broadly described herein, the invention includes a communication hub for controlling an external infusion device. The communication hub includes a first communication interface for transferring information from at least one external device to the communication hub, and a second communication interface for transferring the information received from the at least one external device from the communication hub to the external infusion device. The communication hub is configured to be connected to the external infusion device.

According to another aspect, a system for regulating transport of a fluid into a user includes an external device for providing information used in association with regulating transport of fluid into the user. The system further includes an external infusion device configured to deliver fluid into the user, and a communication hub configured to receive the information from the external device and to be connected to the external infusion device. The communication hub is configured to send information to the external infusion device based on the information received from the external device.

According to yet another aspect, a method for controlling an external infusion device based on information from at least one external device includes transferring information from the at least one external device to a communication hub, and controlling the external infusion device via the communication hub based on the information transferred from the at least one external device to the communication hub.

In still another aspect, a method for enabling an external infusion device to operate based on information transmitted from an external device in at least a first radio frequency and a second radio frequency includes transferring information from the external device to a first communication hub via the first radio frequency, the first communication hub being connected to the external infusion device. The method further includes controlling the external infusion device based on information transferred via the first radio frequency to the first communication hub, and disconnecting the first communication hub from the external infusion device. The method further includes connecting the external infusion device to a second communication hub, and transferring information from the external device to the second communication hub via a second radio frequency. The method further includes controlling the external infusion device based on information transferred via the second radio frequency to the second communication hub.

Aside from the procedural arrangements set forth above, the invention may include a number of other arrangements, such as those explained hereinafter. It is to be understood, that both the foregoing description and the following description are exemplary.

The accompanying drawings are incorporated in and constitute a part of this specification. The drawings illustrate exemplary embodiments of the invention and, together with the description, serve to explain the principles of the invention.

### BRIEF DESCRIPTION OF THE DRAWINGS

FIG. 1 is schematic diagram of an exemplary embodiment of a system for regulating transport of fluid into a user; and

FIG. 2 is a schematic view of an exemplary embodiment of a communication hub.

### DESCRIPTION OF EMBODIMENTS

Reference will now be made in detail to presently preferred embodiments of the invention, examples of which are illustrated in the accompanying drawings.

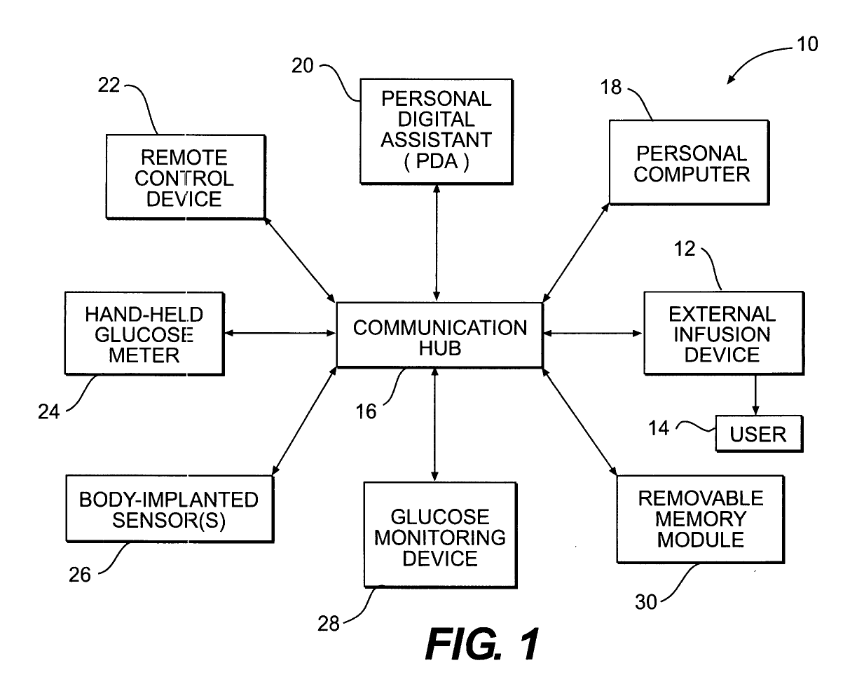

FIG. 1 depicts an exemplary embodiment of a system 10 for regulating transport of fluid into a user 14. The exemplary embodiment schematically depicted in FIG. 1 includes an external infusion device 12, such as, for example, an external infusion pump. External infusion device 12 may be, for example, an external infusion pump for delivering insulin or another treatment to the user 14.

As embodied in FIG. 1, system 10 further includes a communication hub 16 for transferring information between one or more external devices and external infusion device 12. Communication hub is configured in the form of, for example, a relatively low-cost holster configured to support external infusion device 14 on a user's person and/or a leather pouch for supporting external infusion device 14. Communication hub 16 may further include a belt clip configured to attach communication hub 16 to a user's belt.

System 10 further includes one more external devices, and communication hub 16 is configured to transfer information between the one or more external devices and external infusion device 12. For example, external devices include, but are not limited to, a personal computer 18, a personal digital assistant (PDA) 20, a remote control device 22, a hand-held glucose meter 24, one or more body-implanted sensors 26 (i.e., sensors implanted in the body of user 14), and/or a glucose monitoring device 28.

As embodied in FIG. 1, system 10 also includes a removable memory module 30 configured to be inserted and removed from communication hub 16. Removable memory module 30 includes one or more modular flash memory cards (e.g., solid state modules, electromechanical modules, and/or optical modules). Removable memory module 30 is used, for example, by external infusion device 12 for long term storage of diagnostic information, patient history information, external infusion device history information, and/or any other information that is transmitted to communication hub 16 by external infusion device 12 and/or by the one or more external devices in communication with the communication hub 16 via, for example, RF signals. For example, removable memory module 30 is configured to store data associated with operation of communication hub 16, external infusion device 12, and/or external devices, and to be removed from communication hub 16 for insertion into another device configured to enable review of the data.

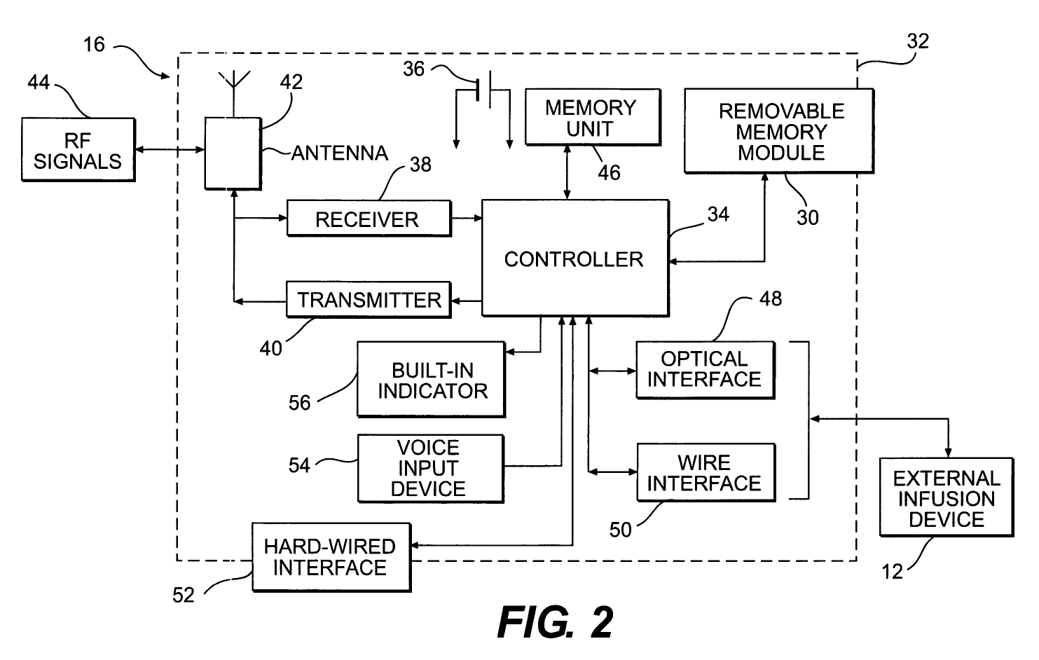

According to some exemplary embodiments, communication hub 16 includes a communication circuit (e.g., an RF communication circuit) configured to communicate with external infusion device 12. For example, circuitry included in communication hub 16 acts as a receiver 38 and/or a transmitter 40 (see FIG. 2), which communicates with external devices such as those mentioned herein, and/or any other device that provides information useful for operating external infusion device 12.

The exemplary embodiment of communication hub 16 schematically depicted in FIG. 2 includes a housing 32. Housing 32 is configured in the form of, for example, a relatively low-cost holster configured to support external infusion device 14 on a user's person, and/or a leather pouch for supporting external infusion device 14. Housing 32 may further include a belt clip configured to attach communication hub 16 to a user's belt.

The exemplary embodiment of a communication hub depicted in FIG. 2 includes a controller 34 and a battery 36 (e.g., a rechargeable battery) for providing power for communication hub 16. Other power sources may be used such as, for example, solar power and/or any other acceptable power source known to those having skill in the art. Communication hub 16 is configured to exchange data and/or control signals between one or more external devices (see, e.g., FIG. 1) and external infusion device 12. For example, controller 34 is operably associated with a receiver 38 and a transmitter 40 that in turn is operably associated with one or more antenna devices 42 for receiving and/or transmitting information to communication hub 16 from one or more external devices. As embodied in FIG. 2, information is transmitted via RF signals 44 and a hard-wired interface 52.

As embodied in FIG. 2, communication hub 16 includes a memory unit 46 and a removable memory module 30 associated with controller 34. Communication hub 16 further includes an interface for transferring information between communication hub 16 and external infusion device 12. For example, communication hub 16 includes an optical interface 48 (e.g., an infra-red interface) and an electrical wire contact interface 50 for transferring information to external infusion device 12. Communication hub 16 also includes a hard-wired interface 52 such as, for example, a USB and/or Fire Wire interface. Communication hub 16 includes communication interface connectors such as, for example, USB, FireWire, and/or similar communication interface connectors, for interfacing communication hub 16 with an external device such as, for example, personal computer 18, PDA 20, hand-held glucose meter 24, and/or glucose monitoring device 28, even when an associated external infusion device 12 has been removed from communication hub 16 (see FIG. 1).

Communication hub 16 further includes a voice input device 54 configured to enable a user to operate system 10 (i.e., communication hub 16 and/or external infusion device 12) via voice command. For example, communication hub 16 is configured to have voice-recognition capability and enables, for example, a visually-impaired user to operate system 10 via vocal commands.

Communication hub 16 also includes a built-in indicator 56, such as, for example, a speaker, a visual read-out, and/or a vibration actuator. Built-in indicator 56 is configured to indicate via, for example, audio, visual, and/or vibratory alarm, the occurrence of certain events, such as, for example, activation of external infusion device 12, and/or it supplements the capabilities inherent in the one or more external devices and/or external infusion device 12.

As embodied in FIG. 2, communication hub 16 is configured such that its RF band frequency may be changed, for example, by changing and/or adjusting the communication circuitry associated with communication hub 16. Some embodiments of communication hub 16 are configured to convert RF signals into optical signals (e.g., infra-red signals), and communication hub 16 communicates with external infusion device 12 via infra-red signals using, for example, an infra-red port. Communication hub 16 also communicates with external infusion device 12 via one or more electrical contacts either instead of via infra-red signals or in addition to via infra-red signals.

According to some embodiments, in addition to, or alternatively to, converting the communication signals in real time to and from the external infusion device 12 from an RF signal into an optical signal and/or an electrical signal (e.g., via an electrical contact), communication hub 16 includes structures and/or features providing communication hub 16 with the ability to perform one or more of the following the functions: ensuring the integrity of RF communications to and from external infusion device 12 (e.g., error checking and/or error recovery); managing collisions from multiple RF signals; operating as a data and/or command buffer, for example, when external infusion device 12 is temporarily disassociated from communication hub 16 (e.g., when external infusion device is removed from communication hub 16 in the form of a holster or pouch) or when the one or more external devices are either disabled or out of range; ensuring the integrity of optical communication to and from external infusion device 12; communicating communication hub-specific data to external infusion device 12 (e.g., battery status of communication hub 16); communicating communication hub-specific data to an external device (e.g., a peripheral communication device); and providing audio and/or vibratory indication for specific events, for example, to supplement capabilities inherent in various external devices that communicate with communication hub 16.

According to some embodiments of communication hub 16, RF circuitry may be configured in one or more forms. For example, the RF circuitry may be of a relatively short-range type, for example, a type that is typically be used to interface with devices in the immediate vicinity of a user 14. Such types typically operate in the U.S. over an ISM band. In some examples, RF circuitry may be configured in a design consistent with computer networking technologies sometimes referred to as “WI-FI” (i.e., IEEE 802.11x or future implementation of such wireless communication protocols). In some further examples, RF circuitry is configured to be of a design consistent with very short range, point-to-point communication protocols, such as, for example, a protocol commonly referred to as “Blue-Tooth.” In other examples, RF circuitry is configured in a longer-range type, for example, so as to operate over a control channel of a cellular telephone network in conjunction with, for example, a technology sometimes referred to as “MicroBurst technology,” an example of which is provided by Aeris Communications, Inc. This type of cellular control channel provides the same or similar coverage as cellular networks, but may experience relatively less traffic and relatively fewer or no system busy conditions. Such technology allows transmission of user and/or external infusion device data, for example, to a remote location for analysis without patient intervention.

As a result of one or more of the exemplary aspects of system 10 and/or communication hub 16, user 14 of external infusion device 12 easily and/or conveniently exchanges one communication hub 16 configured to comply with U.S. communications regulations with another communication hub 16 configured for complying with, for example, European communication regulations when traveling abroad, without substantially affecting operation and/or control of external infusion device 12.

It will be apparent to those skilled in the art that various modifications and variations can be made to the devices and methodology of the present invention. Thus, it should be understood that the invention is not limited to the embodiments discussed in the specification. Rather, the present invention is intended to cover modifications and variations.

1. A communication hub for controlling an external infusion device, the communication hub comprising:
a first communication interface for transferring information from at least one external device to the communication hub; and
a second communication interface for transferring the information received from the at least one external device from the communication hub to the external infusion device,
wherein the communication hub is configured to be connected to the external infusion device.

2. The communication hub of claim 1, wherein the communication hub is configured to be removably connected to the external infusion device.

3. The communication hub of claim 1, wherein the communication hub is configured to communicate with the at least one external device via RF signals.

4. The communication hub of claim 1, wherein the communication hub is configured to communicate with the at least one external device via a hard-wired interface.

5. The communication hub of claim 1, wherein the communication hub is configured to communicate with the external infusion device via an optical interface.

6. The communication hub of claim 1, wherein the communication hub is configured to communicate with the external infusion device via a wire interface.

7. The communication hub of claim 1, wherein the communication hub is configured to receive a removable memory module.

8. The communication hub of claim 1, further comprising a voice input device configured to receive user voice commands.

9. The communication hub of claim 1, further comprising a housing configured to connect the communication hub to the external infusion device.

10. The communication hub of claim 9, wherein the housing is configured to removably connect the communication hub to the external infusion device.

11. The communication hub of claim 1, wherein the communication hub is configured to receive information from at least one of a personal computer, a personal digital assistant, a remote control device, a hand-held glucose meter, a body-implanted sensor, and a glucose monitoring device.

12. The communication hub of claim 1, further comprising an indicator configured to provide a user with information.

13. The communication hub of claim 12, wherein the indicator comprises at least one of a visual indicator, an audible indicator, and a vibratory indicator.

14. A system for regulating transport of a fluid into a user, the system comprising:
an external device for providing information used in association with regulating transport of fluid into the user;
an external infusion device configured to deliver fluid into the user; and
a communication hub configured to receive the information from the external device and to be connected to the external infusion device,
wherein the communication hub is configured to send information to the external infusion device based on the information received from the external device.

15. The system of claim 14, wherein the communication hub is configured to be removably connected to the external infusion device.

16. The system of claim 14, wherein the external infusion device comprises an external infusion pump.

17. The system of claim 14, wherein the external device comprises at least one of a personal computer, a personal digital assistant, a remote control device, a hand-held glucose meter, a body-implanted sensor, and a glucose monitoring device.

18. The system of claim 14, wherein the system is configured to transfer the information from the external device to the communication hub via at least one of RF signals and a hard-wired interface.

19. The system of claim 14, wherein the communication hub is configured to transfer information to the external infusion device via at least one of an optical interface and a wire interface.

20. The system of claim 14, wherein the communication hub comprises a controller configured to receive information from the external device, and based on the information, provide commands to the external infusion device.

21. The system of claim 14, wherein the communication hub and the external infusion device are configured such that the communication hub physically supports the external infusion device.

22. A method for controlling an external infusion device based on information received from at least one external device, the method comprising:
transferring information from the at least one external device to a communication hub; and
controlling the external infusion device via the communication hub based on the information transferred from the at least one external device to the communication hub.

23. The method of claim 22, wherein transferring information from the at least one external device to the communication hub is performed via at least one of RF signals and a hard-wired interface.

24. The method of claim 22, wherein controlling the external infusion device comprises transferring information from the communication hub to the external infusion device via at least one of an optical interface and a wire interface.

25. A method for enabling an external infusion device to operate based on information transmitted from an external device in at least a first radio frequency and a second radio frequency, the method comprising:
transferring information from the external device to a first communication hub via the first radio frequency, the first communication hub being connected to the external infusion device;
controlling the external infusion device based on information transferred via the first radio frequency to the first communication hub;
disconnecting the first communication hub from the external infusion device;
connecting the external infusion device with a second communication hub;
transferring information from the external device to the second communication hub via a second radio frequency; and
controlling the external infusion device based on information transferred via the second radio frequency to the second communication hub.
